(19) United States
(12) Patent Application Publication
(10) Pub. No.: US 2022/0273911 A1
(43) Pub. Date: Sep. 1, 2022

(54) POSITIONING DEVICE AND METHOD

(71) Applicant: HOLISTICK MEDICAL, Paris (FR)

(72) Inventors: Ellen ROCHE, Galway (IE); Matthew KEILLOR, Paris (FR); Philippe POULETTY, Paris (FR); Antoine PAU, Paris (FR); Marco GARD, Borgomasino (IT); Boris WARNACK, Oberwil (CH); Maëlle BRUNEAU, Paris (FR)

(21) Appl. No.: 17/636,578

(22) PCT Filed: Aug. 20, 2019
(86) PCT No.: PCT/IB2019/000915

§ 371 Date: Feb. 18, 2022

(51) Int. Cl. A61M 25/00; A61M 25/01; A61M 25/10

(52) U.S. Cl. CPC A61M 25/0074; A61M 25/0147; A61M 25/10; A61M 2025/0079; A61M 2205/0266; A61M 2025/1093; A61M 2025/1047

(57) ABSTRACT

The invention relates to a positioning device (1) comprising a distal part (8) that is adapted to be delivered through a defect, preferably an opening in a ventricular or atrial septum. It comprises a mechanism to selectively expand the distal part (8), such that before expansion, it can be deployed through said defect, and upon expansion, is mechanically prevented from being retracted through the same defect. Thus, the positioning device (1) is temporarily engaged at the defect site.

The present invention relates to a device and method for deploying a delivery device for a medical implant according to the preamble of the independent claims.

There are numerous medical conditions that are best treated by minimally invasive treatments. As a consequence, a myriad of implantable devices have been proposed in the prior art, many of which can be deployed in a minimally invasive way. For example, closure of a atrial septal defects (ASD) by deploying umbrella-like implants through a catheter have been disclosed by Lock et al. (DOI: 10.1161/01.CIR.79.5.1091).

However, minimally invasive treatments are challenging in that the treatment site is not directly accessible during the treatment. Thus, it is not easily possible for a surgeon to adapt the position or orientation of a delivery device. Similarly, if a delivery device comprises multiple parts, it may be challenging to change the position of one of them while keeping the other at a constant position relative to an implant site. Furthermore, it can be difficult to keep the delivery device at a desired site.

Thus, the object of the present invention is to overcome the drawbacks of the prior art, in particular to provide an easier way of determining the location of a delivery device, in particular in such a way that movements of the delivery device or implant are possible without additional measures to locate the devices.

This and other objects are achieved by the positioning device according to the characterizing portion of the independent claims of the invention.

The positioning device according to the invention is particularly adapted for deploying a delivery device for a medical implant. It comprises a distal part that is adapted to be delivered through a defect, preferably an opening in a ventricular, atrial, or vessel wall. “Distal” shall be understood, in the context of the described device, as the direction away from the operator. It further comprises a mechanism to selectively expand the distal part, enabling an operator to expand the distal part at his discretion. Before expansion, the distal part can be deployed through the defect, whereas after expansion it is mechanically prevented from being retracted through the same defect. Thus, the positioning device is temporarily engaged at the defect site. The distal part is further adapted to be recollapsed and retracted through the medical implant, such that the positioning device can be retracted with the delivery device.

In particular, the device according to the invention may be connected to a delivery device for a medical implant in fixed manner.

It will be understood by the person skilled in the art that the positioning device according to the invention is not designed or adapted to remain in the patient's body. Thus, it differs from medical implants in the prior art in that it is adapted to be removed in an easy way at any time, but in particular during the same procedure as its deployment. The positioning device is, however, of course adapted to be deployed with a medical implant, in particular to facilitate the deployment of a medical implant as the ones known in the prior art.

By being engaged at the defect, the positioning device provides or indicates an anchoring point for the delivery device or implant. Thus, if the position of any part of the delivery device or implant is known relative to the positioning device, its position relative to the defect site can be deduced. In addition, the anchoring at the defect site makes it unnecessary to monitor the exact position of the delivery device relative to the defect site.

Preferably, the positioning device comprises a balloon. In particular, the balloon may be arranged such that in the deployed state, the balloon is located on a different site of the defect than the distal part. In particular, the balloon may be on a different side of a patent foramen ovale (PFO).

This allows an operator or user to provide a counterforce to the balloon. For example, if the balloon is used to press a medical implant against a tissue wall to close the defect, the positioning device can provide a counterforce that aids the deployment.

Preferably, the positioning device comprises at least one wire. The wire has at least a first and a second shape. For example, the first shape may be adapted to allow for easy transfer through a defect, whereas the second shape is adapted to allow for engaging at the defect site.

Preferably, the second shape is substantially planar. This is particularly advantageous to engage the positioning device at the implant site and to provide an even counterforce. Additionally or alternatively, the first shape may be substantially linear to allow for an easy transfer through a defect.

Preferably, the positioning device comprises an actuation mechanism to pull back the distal part of the positioning device in relation the remaining part of the positioning device. The actuation mechanism may, in particular, be a mechanical actuation mechanism. In a particularly preferred embodiment, the actuation mechanism comprises a pull wire. For example, the pull wire may be in operable connection with the distal part of the positioning device, such that pulling of the pull wire directly pulls back the distal part relative to the remaining part of the positioning device.

Preferably, the at least one wire is brought from its first shape into its second shape by actuating the actuation mechanism.

Preferably, the second shape is selected from a group comprising a spiral, a flat disk, and a star.

Preferably, the positioning device comprises a tubular structure, particularly preferably a hypotube.

A hypotube shall be understood as thin, hollow structure comparable to hypodermic needle. In contrast to a hypodermic needle, a hypotube does not necessarily comprise a sharp tip. However, a hypotube may be cannulated throughout as well. A hypotube may comprise or consist of a variety of materials depending on the desired mechanical properties. It is particularly advantageous to use hypotubes made of a biocompatible material. In particular, hypotubes consisting of metals or polymer are well suited for the present application.

Preferably, the tubular structure is made of a shape memory material, even more preferably Nitinol.

Preferably, the tubular structure comprises at least one slit.

Preferably, the at least one slit is arranged such that it forms at least one strut that can extend away from the tubular structure. The strut can also comprise a first and a second shape and engage at the defect site in its second shape.

Preferably, the at least one slit is adapted such that the at least one wire can be deployed through the slit. In particular, the wire may be adapted such that it is arranged within the tubular structure in its first shape and extend away from the tubular structure in its second shape.

Preferably, the at least one slit extends spirally around the circumference and a longitudinal axis of the tubular structure. In such an arrangement, the at least one strut can be extended away from the tubular structure if a part of the tubular structure is rotated. In particular, a distal end of the tubular structure can be rotated around the longitudinal axis of the tubular structure and relative to the tubular structure and/or the positioning device, for example by application of a torque.

The invention also relates to a delivery device.

The delivery device according to the invention comprises a positioning device as described herein. Preferably, the delivery device is mounted concentrically over the positioning device. They can be moved axially relative to each other but they share a common axis, in particular a longitudinal axis. For example, this can be achieved by arranging the positioning device in a dedicated tube in the delivery device. This allows for moving the delivery device along the axis of the positioning device.

The invention further relates to a method of delivering a medical implant in the human body. The method according to the invention is particularly advantageous to deliver a medical implant to an opening in a ventricular or atrial wall. The method comprises the steps of:
Delivering a positioning device, in particular a positioning device as described herein, through an opening in tissue, in particular an opening in an atrial or ventricular wall
Engaging the positioning device at the opening
Delivering the medical implant
Disengaging and removing the positioning device while keeping the implant in the body.

Preferably, the positioning device is retracted trough the medical implant.

The positioning device may comprise a part with a first and a second shape. The step of engaging the positioning device at the opening may comprise bringing said part into its second shape. Preferably, it may be adapted to be brought back into its first shape before retraction.

Optionally, the method may further comprise the step of applying a longitudinal force or a torque, in particular to bring the a part of the positioning device from a first shape into a second shape.

In the following, the invention is described in detail with reference to the following figures, showing:

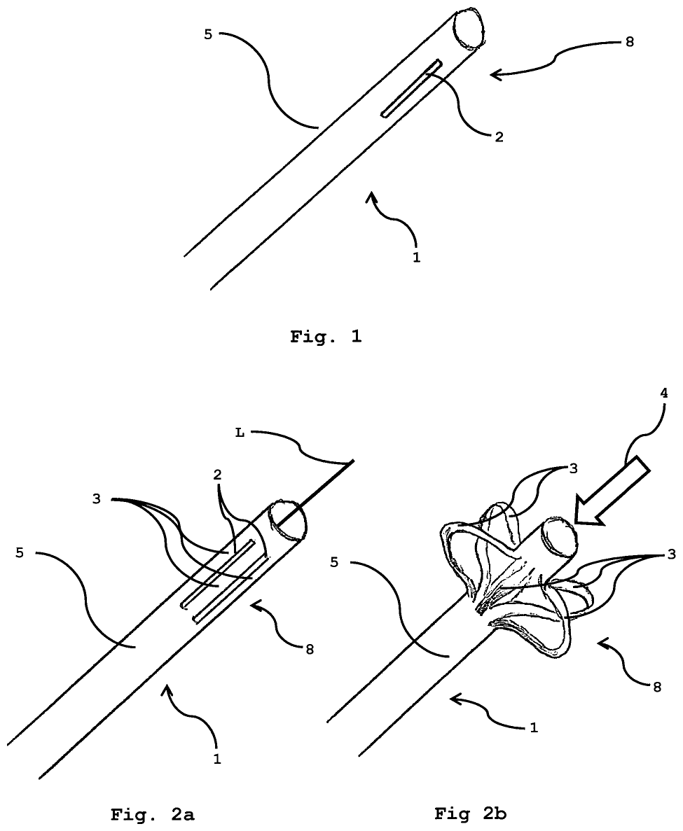

FIG. 1: a schematic depiction of a positioning device comprising a tubular structure.

FIG. 2a-2b: an embodiment of a positioning device in a delivery state and in the engaging state.

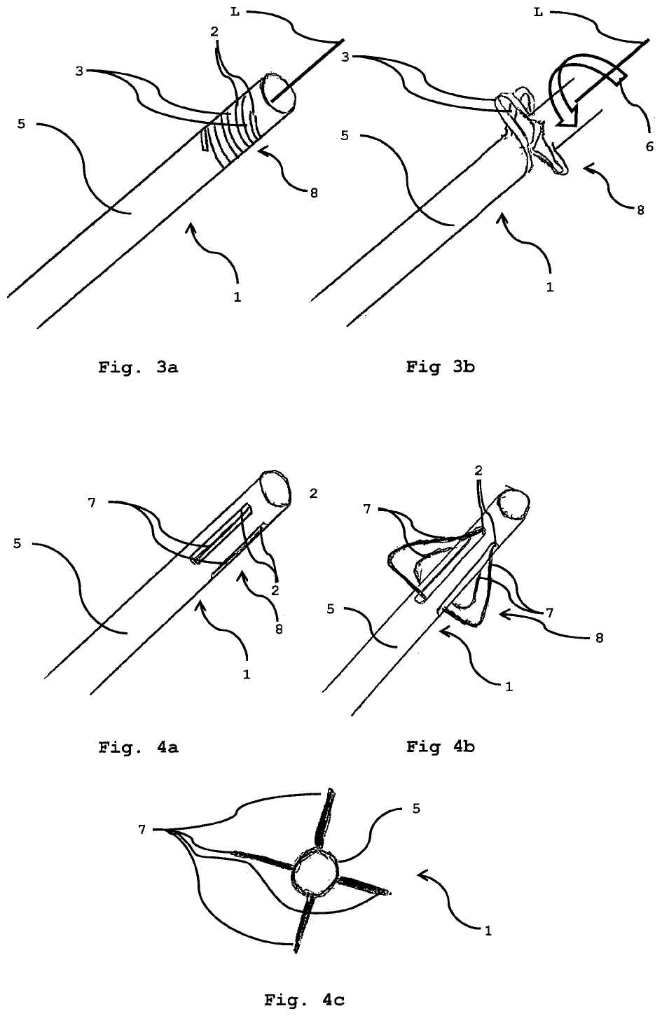

FIG. 3: an alternative embodiment of a positioning device in the deployment state and in the deployed state.

FIG. 4a-4c: another alternative embodiment of a positioning device in the deployment state and in the deployed state, and in a top view.

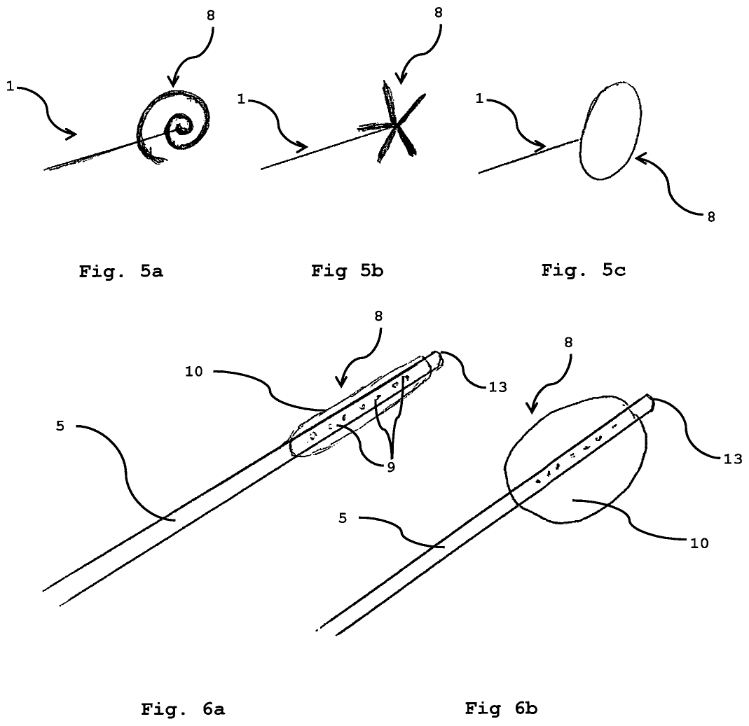

FIG. 5a-5c: different embodiments of a distal part of a positioning device.

FIG. 6a-6b: another alternative embodiment of a positioning device in the deployment state and in the deployed state.

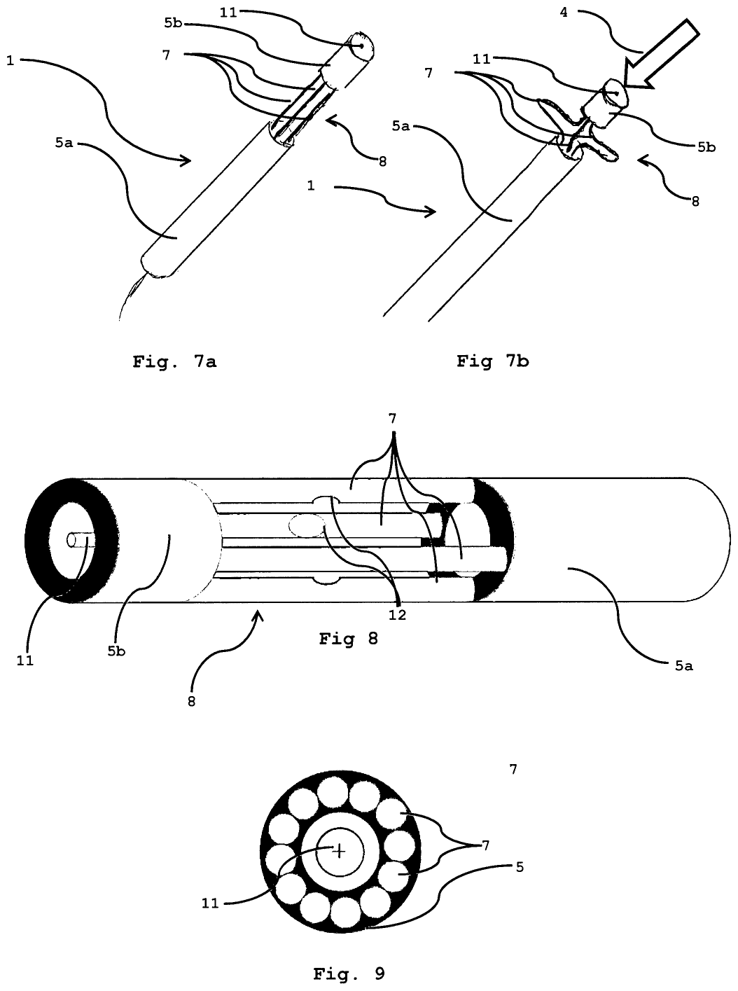

FIG. 7a-7b: another alternative embodiment of a positioning device in the deployment state and in the deployed state

FIG. 8: a detailed view of the distal part of a positioning device as shown in FIG. 7a-b.

FIG. 9: a cross-section along the longitudinal axis of a distal part of a positioning device.

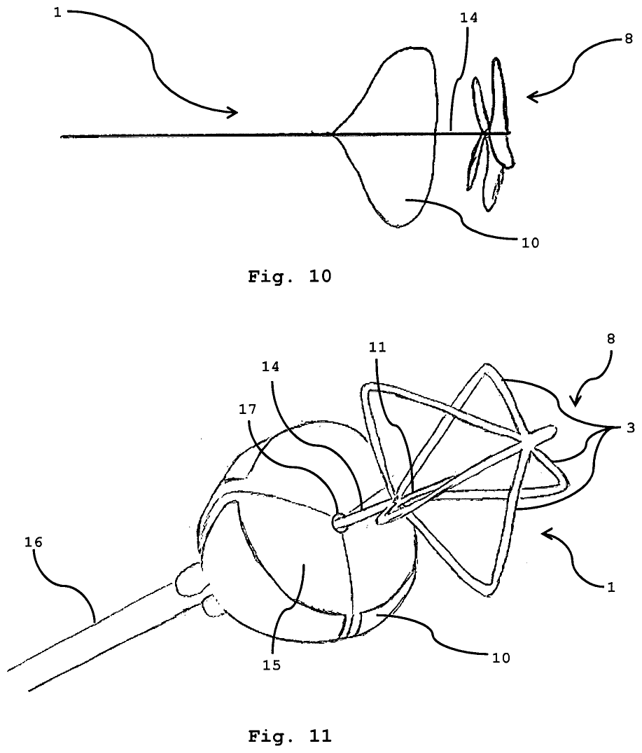

FIG. 10: another alternative embodiment of a positioning device.

FIG. 11 a positioning device in combination with a delivery device.

FIG. 1 shows an embodiment of a positioning device 1 according to the invention. It comprises a tubular structure 5 in the form of a hypotube made of a metallic material. Its distal part 8 comprises one laser-cut slit 2. It is of course possible to use alternative methods to create slits in the hypotube. The slits allow for the deployment of an anchor (not shown) to engage on tissue. In the shown arrangement, however, the anchor is arranged within the hypotube and cannot engage. Thus, the positioning device can be transferred through an opening in an atrial wall. Similarly, retraction of the anchor back into the slit allows for retraction of the device 1 through the same defect.

FIGS. 2a and 2b show an alternative embodiment of a positioning device 1. The positioning device comprises a polymeric hypotube 5 with six slits 2 oriented spread evenly around the circumference of the hypotube 5 and cut along its longitudinal axis L. Only two of the six slits 2 are visible in the shown perspective. The slits form six struts 3 in between them. Thus, the struts 3 and the tubular structure 5 are formed integrally. In FIG. 2a, the positioning device 1 is in a deployment state, meaning that its distal part 8 can be deployed through an opening, for example through a PFO. In FIG. 2b, the positioning device 1 is in its deployed state where its distal part 8 can engage with the tissue to anchor the positioning device. In this embodiment, a longitudinal force 4 is used as an actuating mechanism. Any mechanism that allows for applying such a force 4 can be used, for example a spring, a pull wire, a screw with a nut, or even electric or magnetic means. By applying the force 4, the distal part 8 is moved relative to the remaining part of the positioning device. The struts 3 thus extend away from the positioning device, forming a six arms that extend beyond the positioning device. In this state, the distal part 8 of the positioning device is too large to move through an opening such as a PFO. Thus, if the force is applied after the deployment through said opening, the positioning device 1 is engaged with the wall and prevented from withdrawing through the opening.

FIGS. 3a and 3b show an alternative embodiment of a positioning device 1. The device substantially corresponds to the one described and shown in FIGS. 2a and 2b in that it comprises a hypotube 5 with slits 2 that form struts 3. However, in the present embodiment, the slits 2 extend in a spiral shape along the circumference of the hypotube 5 and its longitudinal axis L. Without actuation, the overall shape of the positioning device 1 substantially corresponds to the shape of the hypotube 5 as is shown in FIG. 3a. The spiral shape of the slits 2 allows for a rotation 6 of the distal part 8 with respect to the positioning device 1 to make the struts 3 extend. Thus, instead of a longitudinal force as shown in FIG. 2b, the application of a torque is the actuation mechanism in this embodiment. Upon rotation 6, the struts 3 extend an the distal part 8 of the positioning device 1 thus engages in the opening.

FIG. 4a-4c show yet an alternative embodiment of a positioning device 1 according to the invention. The device comprises a tubular structure 5, here in the form of a metallic hypotube made of surgical steel. It further comprises four slits 2 that are spread evenly around the circumference of the hypotube 5. Four wires 7 are arranged within the wall of the hypotube such that they are substantially parallel to and overlap with the slits 2 in the deployment state shown in FIG. 4a. In the present embodiment, the wires consist of a nickel-titanium alloy with shape memory properties. Thus, for example by an increase in temperature due to the exposure to human body temperature, the wires 7 are brought into their second shape and extend through the slits 2 away from the positioning device 1 and the hypotube 5. Thus, the distal part 8 can engage at the wall surrounding the opening and anchor the positioning device 1. FIG. 4c shows a top view (from the distal part in a proximal direction) of the positioning device 1 in its deployed state. The wires 7 extend away from the hypotube 5 and are spread evenly in 90° angles around the circumference of the positioning device.

FIGS. 5a, 5b, and 5c schematically show different embodiments of a distal part 8 of positioning device 1. FIG. 5a shows a distal part comprising a spiral-shaped wire. This arrangement is partitularly advantageous because the spiral shape allows for a simple compression within an outer sheath such as a hypotube or another tubular structure. In particular, if the distal part is made of an elastic material such a Nitinol, it can be compressed like spiral spring. In addition, the shape memory properties can be capitalized on to, for example, bring the spiral back into its first shape such that it can be retracted through the medical implant and the opening. It will be understood by the person skilled in the art, however, that the shapes of the distal part depicted here are not restricted to a particular actuation mechanism but merely show different embodiments of a distal part. As such, the spiral shown in FIG. 5a may also be extended by other mechanical or electrical forces. Additionally or alternatively, it may be arranged as a spiral spring wherein the relaxed shape is the first shape for deployment, and a force is necessary to extend it. Such an arrangement would regain its original shape simply by stopping the application of a force, facilitating the retraction of the distal part 8.

FIG. 5b shows a different embodiment 1 of a distal part 8 with a star shape. This embodiment offers the advantage that the five bars forming the star can be mounted rotatably along an axis orthogonal to the longitudinal axis of the positioning device 1. For example, the bars forming the stars can arranged in a parallel manner to the longitudinal axis while deploying and orthogonally in the deployed state. It may also be made of more or less bars than the five shown here, depending on the spatial constraints of the application it intended for.

FIG. 5c shows yet another embodiment wherein the distal part 8 comprises a flat disk made of a polymer. The polymer may be a shape memory polymer and/or a flexible polymer for easier deployment. It is of course possible to make the flat disk out of a different material, for example a metal, with similar mechanical properties.

FIGS. 6a and 6b show an embodiment of a positioning device 1 comprising a balloon 10 on its distal end 8. The positioning device 1 also comprises a tubular structure 5. However, the tubular structure has a closed ending 13 such that it does not stand in fluid connection with its surrounding. Instead, the tubular structure comprises several holes 9 on its side wall. The balloon 10 is arranged around the side walls of the tubular structure such that all holes 9 are within the balloon. The balloon is sealed such that its inside is not in fluid connection with its outside. FIG. 6a shows the deployment state of the device with a substantially linear outer shape that can be deployed through an opening such as a PFO. FIG. 6b shows the deployed state wherein a fluid has been pressed through the holes 9 and filled up the balloon 10. The extended diameter of the balloon prevents it from being retracted through the opening and thus engages the positioning device. The balloon 10 may be elastic, leading to more flexibility for the surgeon to fill it up to a desired size by applying more pressure on the fluid. However, it is of course also possible to use a non-elastic material. In this case, the extended size of the balloon 10 cannot be changed.

FIGS. 7a and 7b show yet another embodiment of a positioning device 1 according to the invention. The tubular structure 5a, 5b is divided in two parts, one of which 5b is arranged at the distal part 8. The two parts of the tubular structure are connected by four elastic wires 7. Furthermore, a pulling wire 11 is connected to the distal part 8, in particular two the distal part of the tubular structure 5b. Pulling of the pulling wire moves the distal part 8 closer relative to the remaining part of the positioning device 1, thus bending the wires that form partial loops extending from the positioning device. These partial loops are adapted to engage the tissue around the opening, thus positioning the positioning device 1. The positioning device 1 can easily be brought into the initial position by releasing the pull wire 11. The elastic force of the bent wires 7 then pushes the distal part 8 back to its original position. It will be understood by the person skilled in the art that a pull wire 11 is a very well suited mechanism to actuate the bending of the wires 7. However, any other mechanism such as a screwing mechanism, a spring mechanism, a hydraulic mechanism, or electric or magnetic means may also be employed to move the distal part 8 closer to the positioning device 1.

FIG. 8 shows a more detailed depiction of the distal part 8 of the positioning device 1 shown in FIGS. 7a and 7b. The tubular structure 5a, 5b comprises two parts connected by four wires 7 and a pull wire 11. In this illustration, an additional feature of the wires 7 is well visible. In order to facilitate the bending of the wires 7 upon pulling of the pull wire 11, the wires 7 comprise one groove 12 each. Here, the groove has the form of tangential and spherical cut on the inside of the wires 7. However, other shapes may also be employed. For example, a triangular cut may be more suitable for a particular application. Additionally or alternatively, a simple cut on the outside of the wires may also be employed. The grooves 12 reduce the necessary force to pull the distal part 8 back relative to the positioning device 1. In addition, they allow for a better control of where the bending of the wire is most pronounced. By using several grooves, it is also possible to control the shape of the wires 7 upon actuation of the pull wire 11. For example, two grooves 12 per wire 7 may lead to rectangular shape instead of a spherical shape of the wires 7. The diameter of the positioning device 1 shown here is approximately 1 mm, but may be larger or smaller based on the defect that is to be treated. The diameter of the wires 7 is 0.15 mm each, and the length of the wires 7 connecting the distal part with the remaining part of the positioning device 5a is 1 cm. Of course, these values may be changed and adapted to a particular application and should not be understood as limiting the invention.

FIG. 9 shows a cross-section of the distal part 8 similar to the one shown in FIGS. 7a, 7b, and 8. In contrast to the devices shown in those figures, the embodiment shown here comprises twelve wires 7 instead of four. This allows for a more robust engaging while less force is applied on the tissue by each individual wire 7. Thus, this embodiment is particularly advantageous for sensitive tissue. Beyond the number of wires 7, this embodiment is identical to the ones described and referenced above. It also comprises a pull wire 11 to pull back that is connected to the tubular structure 5. Similarly, the wires 7 are connected to the tubular structure 5.

FIG. 10 shows a different embodiment of a positioning device 1 comprising a balloon 10. Here, the balloon 10 is arranged such that in the deployed state, it is on a different side of relative to the position 14 opening and the wall that is treated than the distal part. Here, the distal part 8 is a star-shaped wire arrangement similar to the one described in FIG. 5b. However, the distal part 8 could be any distal part as described herein, of course. This particular arrangement allows the distal part 8 to act as a counterforce to the balloon 10. For example, if the positioning device 1 is used to aid the deployment of a patch-like medical implant, the balloon 10 could apply a force onto said medical implant while the distal part pushes it back.

FIG. 11 shows a combination of a delivery device 16 and a positioning device 1. The delivery device 16 comprises a balloon 10 to which an implant 15 can be attached. Here, the delivery device 16 comprises an inner tube that extends through the balloon 10 and yields in a hole 17 on the balloon surface. This enables the deployment and retraction of the positioning device 1 regardless of the inflation state of the balloon. However, it will of course be understood by the person skilled in the art that the delivery device can be adapted in any other way to be combined with a positioning device. As such, the shown embodiment shall not be understood as limiting the invention. The positioning device 1 is similar to the one shown in FIGS. 2a and 2b. It comprises a metal tube, in this case made from titanium, and laser-cut struts 3. On the inside of the tube, there is a pull wire 11. By pulling of the pull wire 11, the distal part 8 of the positioning device is moved along the longitudinal axis of the delivery device 16 in the direction of the balloon 10. This causes the struts 3 to extend. The shown system is particularly advantageous to treat a defect in an atrial wall. The positioning device is adapted to be delivered through said defect (not shown) and engages the tissue when the struts 3 extend away from the longitudinal axis. The area 14 of the positioning device can be located in said atrial defect during delivery. The implant 15 can be attached to the defect to close it, after which the positioning device 1 can be retracting by releasing the pull wire to bring the positioning device 1 back to a substantially linear shape and retracting it through the hole 17 in the implant 15.

1.-15. (canceled)

16. A positioning device for deploying a delivery device for a medical implant, the positioning device comprising:
a distal part that is adapted to be delivered through a defect, wherein the positioning device comprises a mechanism to selectively expand said distal part, said distal part being adapted such that before expansion, it can be deployed through said defect, and upon expansion, is mechanically prevented from being retracted through the same defect, such that the positioning device is temporarily engaged at the defect site; and
the distal part adapted to be retracted through said medical implant such that upon the deployment of said implant, the positioning device can be retracted with the delivery device.

17. The positioning device according to claim 16, wherein the positioning device comprises a balloon.

18. The positioning device according to claim 16, wherein the distal part of the positioning device comprises at least one wire, said wire having a first and a second shape.

19. The positioning device according to claim 18, wherein the second shape is substantially planar.

20. The positioning device according to claim 18, wherein the positioning device comprises an actuation mechanism to pull back the distal part of the positioning device in relation to the positioning device.

21. The positioning device according to claim 20, wherein the positioning device is adapted such that the at least one wire is brought from its first shape into its second shape by actuating the actuation mechanism.

22. The positioning device according to claim 18, wherein the second shape is selected from a group comprising a spiral, a flat disk and a star.

23. The positioning device according to claim 16, comprising a tubular structure.

24. The positioning device according claim 23, wherein the tubular structure is made of a shape memory material.

25. The positioning device according to claim 23, wherein the tubular structure defines at least one slit.

26. The positioning device according to claim 25, wherein the at least one slit is arranged such that it forms at least one strut that can extend away from the tubular structure.

27. The positioning device according to claim 18, wherein the tubular structure defines at least one slit.

28. The positioning device according to claim 27, wherein the at least one slit is adapted such that the at least one wire can be deployed through said slit.

29. The positioning device according to claim 23, wherein the at least one slit extends spirally around the circumference and a longitudinal axis (L) of the tubular structure,such that the at least one strut can be extended away from the tubular structure,by a rotation of a part of the tubular structure,.

30. A catheter device, characterized in that the catheter device comprises a positioning device according to claim 16.

31. A method of delivering a medical implant in the human body, comprising the steps:
delivering a positioning device, through an opening in a wall;
engaging the positioning device at the opening;
delivering the medical implant; and
disengaging the positioning device.
